(19) United States
(12) Patent Application Publication
(10) Pub. No.: US 2011/0166478 A1
(43) Pub. Date: Jul. 7, 2011

(54) One Touch Laser Lancing Type Blood Glucose Measurement Device, Strip and the Method Using It

(75) Inventor: Kee Jung Choi, Daejeon (KR)

(73) Assignee: ISOTECH CO., LTD, Daejeon (KR)

(21) Appl. No.: 13/061,368

(22) PCT Filed: Aug. 29, 2008
(86) PCT No.: PCT/KR2008/005067

§ 371 Date: Feb. 28, 2011

(30) Foreign Application Priority Data
Aug. 27, 2008 (KR) 10-2008-0083705

(51) Int. Cl. A61B 5/151

(52) U.S. Cl. 600/583

(57) ABSTRACT

A one touch laser lancing type blood glucose measurement device, a strip, and the method using it. In particular, the invention relates to a one touch laser lancing type blood glucose measurement device, a strip, and the method using it, which performs blood gathering and blood glucose measurement at the same time, using laser beams generated in a laser module. The laser beams are irradiated into a finger passing a hole of a strip with a focusing lens converging the laser beams, a protect window, and the hole to gather blood, then the blood is absorbed into the strip for measuring blood glucose.

### CROSS-REFERENCE TO RELATED APPLICATIONS

This application is the U.S. national phase of International Patent Application No. PCT/KR2008/005067 filed Aug. 29, 2008, which claims the benefit of Korean Application No. 10-2008-0083705 filed Aug. 27, 2008, the entire content of which is incorporated herein by reference.

### BACKGROUND

1. Field of the Invention

The invention relates to a one touch laser lancing type blood glucose measurement device, a strip, and the method using it. In particular, the invention relates to a one touch laser lancing type blood glucose measurement device, a strip, and the method using it, which performs blood gathering and blood glucose measurement at the same time, using laser beams generated in a laser module. The laser beams are irradiated to a finger passing a hole of a strip with a focusing lens converging the laser beams, a protect window, and the hole to gather blood, then the blood is absorbed into the strip for measuring blood glucose.

2. Description of the Related Art

Recently, the International Diabetes Federation (IDF) warned that diabetics number over about 360 millions around the globe, and the number of diabetics increases about 8.6% every year, therefore solutions by the government are required.

For diagnosing diabetes, the density of glucoses in blood plasma of blood components is measured, and management of blood sugar is very important.

Generally, blood-gathering and blood glucose measurement devices prick the skin surface by using a lancing device with a lancet having a metal needle shape. Thus, diabetics feel pain and have scars, and a superfluous amount of blood is gathered and dropped onto a strip end. Then, a strip is inserted into a blood glucose measurement device separately formed, to measure blood sugar level.

As described above, for measuring blood sugar level, the lancet, the lancing device, the blood glucose measurement device, the strip, and alcohol wipes are needed.

However, due to excessive TV watching and irregular diet habits, the number of children suffering from diabetes increases. The lancet having the needle causes pain and fear in children and adults.

In addition, a diabetic relying on insulin injections who measures the blood sugar level 2 to 4 times every day must have a blood glucose measurement device, but it is onerous to carry the various components of the blood glucose measurement device.

To solve these problems, a blood glucose measurement device apparatus comprising a lancet, a lancing device and a blood-gathering device using laser beams has been developed. Blood is gathered using laser beams in one end of the apparatus and is dropped onto the strip, then the strip is inserted into a strip connector to measure blood sugar level in another end of the apparatus.

However, even though the apparatus has both a blood-gathering function and a blood sugar level measuring function, blood is gathered by inserting a disposal cap into one end of the apparatus, while the strip is inserted into another end of the apparatus for measuring blood sugar level. Thus, using the apparatus is not easy. To avoid pollution of the blood glucose measurement device, a disposal cap covered with a film is used, but has to be carried.

Since the apparatus has a blood glucose measurement device and a blood-gathering device on opposite ends, the volume of the apparatus increases. When the diabetic relying on insulin injections is a child, controlling the apparatus is difficult due to the large volume.

### SUMMARY

An embodiment of the invention provides a one touch laser lancing type blood glucose measurement device, a strip, and the method using it performing a blood-gathering process, a process of sucking blood with the strip, and a process of measuring blood glucose level by a one button operation at the same time in a blood glucose measurement device and the strip.

Another embodiment of the invention provides a one touch laser lancing type blood glucose measurement device, a strip, and the method using it with decreased volume by combining a blood-gathering device and a blood glucose measurement device.

Another embodiment of the invention provides a strip forming a film on an upper end of a strip hole in order to avoid pollution of the device.

A one touch laser lancing type blood glucose measurement device according to the invention comprises a laser module generating laser beams and irradiating the laser beams in one direction; a focusing lens converging the laser beams generated by the laser module and disposed in a laser irradiation direction; a protect window disposed in a laser direction progressing through the focusing lens and protecting the focusing lens from foreign bodies; a controlling unit disposed on a position close to a lateral side of the laser module and controlling a blood measure and the laser beams; a strip connector connected to the controlling unit and formed in a position on which a strip hole and the protect window are coincided with each other, and inserting a strip; a body unit containing the laser module, the focusing lens, the protect window, the controlling unit, and the strip connector; a interlock disposed on the outside of the body unit, positioned on the upper or lower side of the strip inserted into the strip connector, forming a hole, and changing the laser module by pressing with a finger into a ready state; and a button unit formed on a lateral side different form or an opposite side to the interlock holder and operating the laser module.

The invention further comprises a finger interlock formed on the outside of the body unit, preventing the laser beams irradiated from the laser module from being exposed to the exterior, and having a shape, which a finger is inserted into and a first sensor making a user sense the finger interlock's existence.

The invention further comprises a second sensing unit pressing the interlock holder to sense the ready state of the laser module.

In a strip used in a blood glucose measurement device according to the invention, the strip comprises a reagent unit; a electrode unit inserted into strip connector to transfer blood information to the blood glucose measurement device; a strip hole passing laser beams irradiated from the laser module to irradiate the laser beams; and a polypropylene film included on the upper of the strip hole to prevent the blood glucose measurement device from becoming polluted.

The reagent unit measures a glucose from among the group consisting of a ferment, anti-insulin, and HbAIc.

A method for measuring blood glucose using a one touch laser lancing type blood glucose measurement device according to the invention comprises a first process of inserting a strip into a strip connector; a second process of inserting a finger into an upper of an interlock holder and into the inner of the finger lock holder; a third process of changing into a ready state by pressing the interlock holder; a fourth process of operating a laser module using a button module to generate laser beams, after the change into the ready state; a fifth process of converging the generated laser beams into a focusing lens, passing a protect window, a strip hole, and an interlock holder, and then irradiating the laser beams into the inserted finger; a sixth process of causing blood oozed from the finger due to the irradiating laser beams to be absorbed into the strip; a seventh process of transmitting data regarding the absorbed blood to a controlling unit; and an eighth process of outputting analyzed data regarding the absorbed blood from the controlling unit to a displaying unit.

A one touch laser lancing type blood glucose measurement device, a strip, and the method using it according to the invention performs a blood-gathering process, a process of sucking blood with the strip, and a process of measuring blood glucose level by a one button operation at the same time in the blood glucose measurement device and the strip.

The invention is a mixed construction having a blood-gathering device and a blood glucose measurement device installed on one side, thereby decreasing the volume of the device. Thus, the one touch laser lancing type blood glucose measurement device is easily portable.

The invention blocks dust etc. absorbed on the focusing lens by irradiating high temperature laser beams generated in blood-gathering by laser beams and exactly measuring the blood sugar level using a small amount of blood.

### BRIEF DESCRIPTION OF THE DRAWINGS

FIG. 1 is a diagram representing a one touch laser lancing type blood glucose measurement device according to the invention.

FIG. 2 shows a diagram representing blood-gathering using a one touch laser lancing type blood glucose measurement device according to the invention.

FIG. 3 shows that blood gathered using a one touch laser lancing type blood glucose measurement device according to the invention is absorbed into a strip.

FIG. 4 is a diagram showing a strip used in a one touch laser lancing type blood glucose measurement device according to the invention.

FIG. 5 is a flow chart of a blood glucose measurement method using a one touch laser lancing type blood glucose measurement according to the invention.

### DESCRIPTION OF THE PREFERRED EMBODIMENTS

Hereinafter, embodiments of the invention will be described in detail referring to the accompanying drawings.

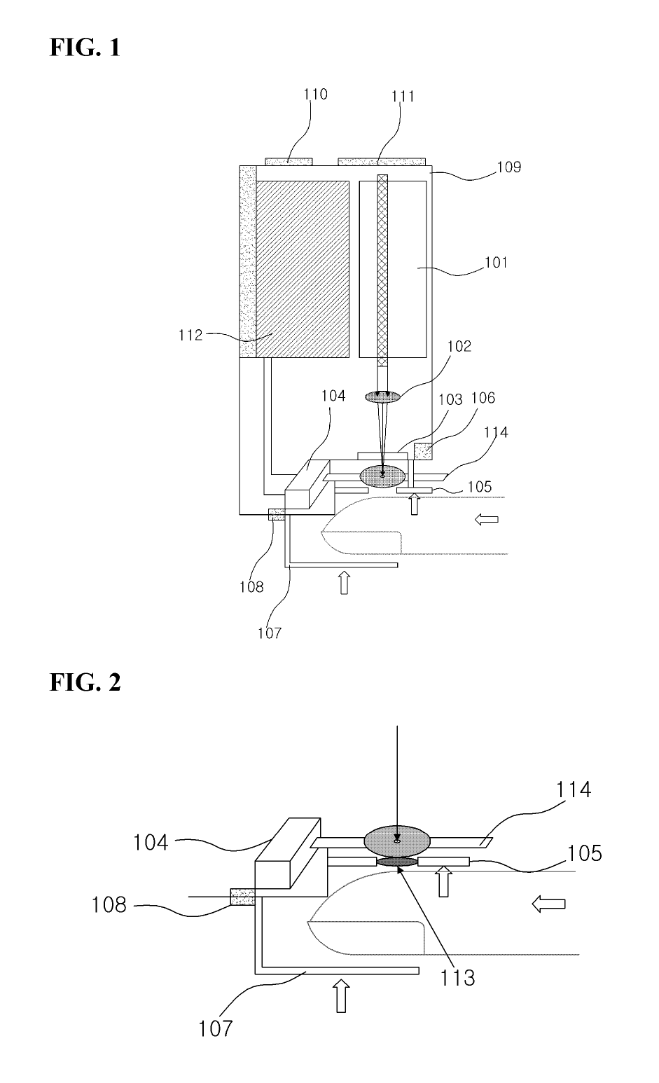

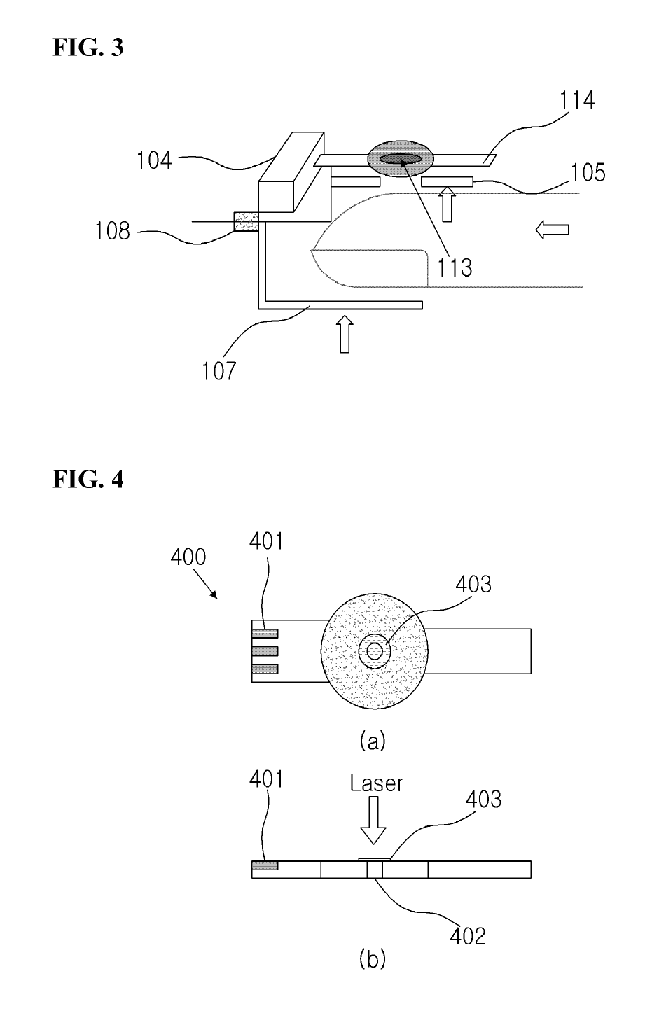

FIG. 1 is a diagram representing a one touch laser lancing type blood glucose measurement device according to the invention, FIG. 2 shows a diagram representing blood-gathering using a one touch laser lancing type blood glucose measurement device according to the invention, and FIG. 3 shows that blood gathered using a one touch laser blood lancing type glucose measurement device according to the invention is absorbed into a strip.

As shown in FIGS. 1 to 3, a one touch laser lancing type blood glucose measurement device comprises a laser module 101, a focusing lens 102, a protect window 103, a controlling unit 112, a strip connector 104, a body unit 10, a interlock holder 105, a second sensing unit 106, a button unit 110, a finger interlock 107, a first sensing unit 10, and a displaying unit 111.

The laser module 101 generates laser beams and irradiates the laser beams in a single direction to help a user gather blood in a user's finger. The laser beams generated in the laser module 101 are an Er:YAG laser technique and may gather the blood 113 in skin without a scar. The laser beams also have a wavelength of 2.94 μm that has a strong absorbing power with respect to water and leaves no poisons in the body.

In the invention, the Er:YAG laser technique is described as an embodiment. However, the invention is not limited to the Er:YAG laser technique and a laser technique having the characteristics equal to the Er:YAG laser technique and not harmful to the body may be used.

The focusing lens 102 converges the laser beams into a limited pricked skin part. In more detail, the focusing lens 102 is an element that converges the laser beams progressing in a predetermined direction to blood-gathering object skin, that is, a finger with an accurate focus. Thereby, the focusing lens 12 may prick to make a hole having a diameter of 0.1 mm to 0.5 mm and a depth of 0.2 mm to 4 mm. However, to avoid painful sores by stimulation of nerve cells into the true skin, it is preferable that the focusing lens 12 may prick to make a hole having a depth of 0.5 mm or less and diameter of 0.3 mm or less.

The focusing lens 102 may also use a convex lens or an aspherical lens accurately to focus the laser beams to the pricked skin part.

The protect window 103 protects the focusing lens 102 from foreign bodies in the direction in which the laser beams from the focusing lens 102 progress. The protect window 103 is spaced apart from the focusing lens 102 by a predetermined distance in the laser progressing direction.

The controlling unit 112 controls a blood sugar level measurement of blood input into a position close to a lateral side of the laser module 101 and the laser beams.

The strip connector 104 connects to the controlling unit 112, but the strip connector 104 is formed in a position on which a strip hole and the protect window 103 are coincided with each other and inserts a strip 114. The used strips 114 and 400 will be described in detail later, referring to FIG. 4. The strip connector 104 obtains data of the blood 113 in the strip 114 to transfer the data to the controlling unit 12.

The body unit 109 contains the laser module 101, the focusing lens 102, the protect window 103, the controlling unit 112, and the strip connector 104. At this time, the strip connector 104 is contained into the body unit 109, but it is evident that a gate for inserting the strip 114 is exposed to the outside from the body unit 109.

The interlock holder 105 is disposed on the outside of the body unit 19, and positioned on the upper or lower side of the strip 114 inserted into the strip connector 104. The interlock holder 105 forms a hole. The hole disposes the strip hole 402 formed in the strip, the protect window 103, and the focusing lens 102 on a straight line, and then is pressed by a finger. Thereby, a state of the laser module 101 is changed into a ready state. At this time, the strip hole 402 is shown in FIG. 4.

When the state of the interlock holder is changed into the ready state, laser beams may be generated using a button. However, when the state of the interlock holder is not changed into the ready state, laser beams may not generated regardless of a state of the button, to protect the user. The Er:YAG laser technique is not harmful to the body. However, when the laser beams generated by the Er:YAG laser technique irradiate the eyes, the eyes may be irreversibly damaged. Therefore, the invention may prevent accidents by changing the state of the interlock holder.

The second sensing unit 106 presses the interlock holder 105 to sense the ready state of the laser module.

The finger interlock 107 formed on the outside of the body unit 109, prevents laser beams generated in the laser module 101 from being exposed to the exterior, and has a shape, which a finger is inserted into. Since the finger interlock 107 may be inserted inward, it may be easy to take custody of the blood glucose measurement device of the invention.

The first sensing unit 108 senses whether the finger interlock 107 is opened. When the finger interlock 107 is damaged, laser beams may be exposed to the exterior. Thus, when the finger interlock 107 is damaged, the first sensing unit 108 operates to stop the operations of the blood glucose measurement device, to function as a safety device.

The button unit 110 is formed on a different position from the interlock holder 105 to operate the laser module 101. The button unit 110 is able to operate only when the interlock holder 105 is in the ready state, for the user's safety.

The displaying unit 111 outputs data measured by the operating of the laser module 101 by the button unit 10 and by the transferring the blood 113 obtained by the strip 114 to the controlling unit 112 through the strip connector 104.

As described herein, since the blood-gathering device and the blood glucose measurement device are on one side and complexly formed, there is an advantage that the blood-gathering and the blood sugar level measurement are performed by a one button operation.

FIG. 4 is a diagram showing a strip used in a one touch laser lancing type blood glucose measurement device according to the invention.

As shown in FIG. 4, a strip 400 inserted into the strip connector used in the invention comprises an electrode unit 401, a strip hole 402, a reagent unit (not shown), and a PP film 403.

The electrode unit 401 is inserted into the strip connector 104 to transfer blood information to the controlling unit of the blood glucose measurement device.

The strip hole 402 passes laser beams generated by the laser module to irradiate the laser beams to transfer a finger and the blood of the finger to the reagent unit. At this time, preferably, a diameter of the strip hole 4 is 3 mm, but the invention is not limited to that.

It is preferable that the reagent unit (not shown) may measure a glucose from among the group consisting of a ferment, anti-insulin, and HbAIc. In addition, the invention using the strip may measure at least one of ketone and cholesterol.

The PP (poly propylene) film 403 is included on the upper of the strip hole to prevent the blood glucose measurement device from becoming polluted. It is preferable that the PP film 403 has a thickness of 0.05 mm to 0.15 mm, and it is more preferable that the PP film 403 has a thickness of 0.1 mm.

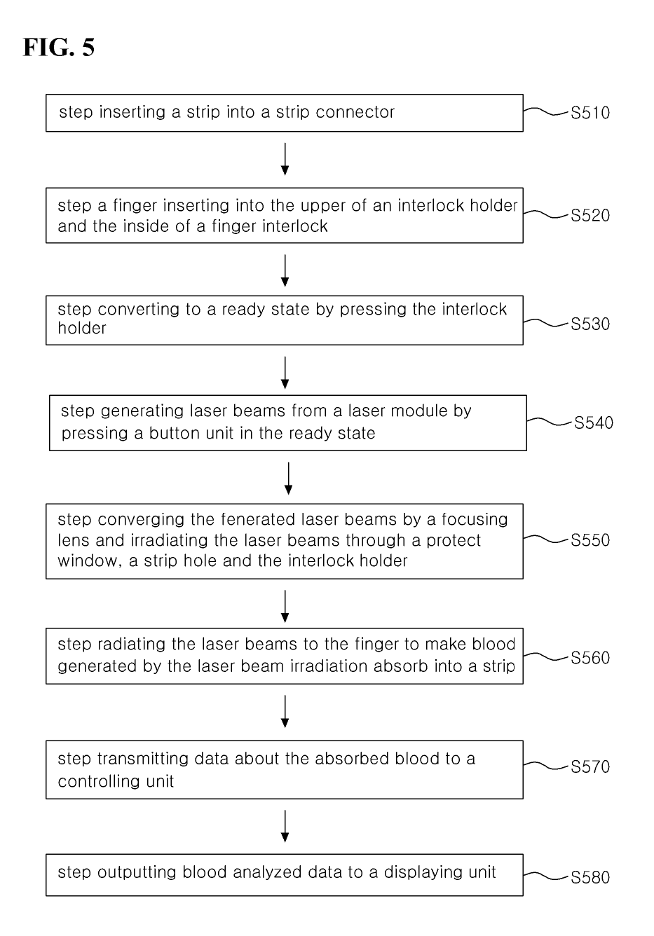

FIG. 5 is a flow chart of a blood glucose measurement method using a one touch laser lancing type blood glucose measurement according to the invention.

As shown in FIG. 5, the blood glucose measurement method using a one touch laser lancing type blood glucose measurement according to the invention will be described below.

First, the strip is inserted into the strip connector (S510). At this time, the strip hole is coincided with the focus lens and the protect window by the strip inserted into the strip

Sequentially, a finger is inserted into the upper of the interlock holder and the inner of the finger interlock (S520).

Sequentially, the interlock holder is pressed to be changed into a ready state (S530). At this time, in being charged into the ready state, the displaying unit displays the time when the button unit is pressed.

Sequentially, when the button unit is pressed in the ready state, laser beams are generated by the laser module (S540). At this time, the ready state is displayed in the displaying unit, and a user presses the button unit to operate the laser module.

Sequentially, the generated laser beams are converged onto the focusing lens and irradiated through the interlock holder into the finger (S550). The laser beams irradiated into the finger cause blood to ooze from the finger.

The principle of the blood oozing caused by the laser beams is as below.

When the laser module generates laser beams of a high temperature to irradiate into the finger, the skin of the finger is burned by the laser beams to stimulate capillaries of the inner periphery and burst them. Thereby blood oozes from the finger.

It is preferable that the temperature of the laser beams is in the range from 1000° C. to 1300° C. Since the laser beams have a high temperature, it is possible to perform the blood-gathering without a disinfection operation.

Sequentially, the blood gathered by the irradiation of the laser beams is absorbed into the strip (S560).

Sequentially, data regarding the blood absorbed into the strip is transferred to the controlling unit. At this time, a glucose from among the group consisting of a ferment, anti-insulin, and HbAIc may be measured (S570).

Finally, the controlling unit outputs analyzed data regarding the blood to the displaying unit (S580).

While this invention has been described in connection with what is considered to be practical exemplary embodiments, it is to be understood that the invention is not limited to the disclosed embodiments, but, on the contrary, is intended to cover various modifications and equivalent arrangements included within the spirit and scope of the appended claims.

### DESCRIPTION OF REFERENCE CHARACTERS

101: laser module
103: protect window
105: interlock holder
107: finger interlock
109: body unit
111: displaying unit
113: blood
401: electrode unit
403: polypropylene film

1. A one touch laser lancing type blood glucose measurement device comprising:
a laser module generating laser beams and irradiating the laser beams in one direction;
a focusing lens converging the laser beams generated from the laser module and disposed in a laser irradiation direction;
a protect window disposed in a laser direction progressing through the focusing lens and protecting the focusing lens from foreign bodies;
a controlling unit disposed on a position close to a lateral side of the laser module and controlling a blood measure and the laser beams;
a strip connector connected to the controlling unit and formed in a position on which a strip hole and the protect window are coincided with each other, and inserting a strip;
a body unit containing the laser module, the focusing lens, the protect window, the controlling unit, and the strip connector;
a interlock disposed on the outside of the body unit, positioned on the upper or lower side of the strip inserted into the strip connector, forming a hole, and changing the laser module by pressing with a finger into a ready state; and
a button unit formed on a lateral side different from or an opposite side to the interlock holder and operating the laser module.

2. The device of claim 1, further comprising a finger interlock formed on the outside of the body unit, preventing the laser beams irradiated from the laser module from being exposed to the exterior, and having a shape, which a finger is inserted into and a first sensor making a user sense the finger interlock's existence.

3. The device of claim 1, further comprising a second sensing unit pressing the interlock holder to sense the ready state of the laser module.

4. A strip used in a blood glucose measurement device, the strip comprising:
a reagent unit;
an electrode unit inserted into strip connector to transfer blood information to the blood glucose measurement device;
a strip hole passing laser beams irradiated from the laser module to irradiate the laser beams; and
a polypropylene film included on the upper of the strip hole to prevent the blood glucose measurement device from becoming polluted.

5. The strip of claim 4, in which the reagent unit measures a glucose from among the group consisting of a ferment, anti-insulin, and HbAIc.

6. A blood glucose measurement method comprising:
a first process of inserting a strip into a strip connector;
a second process of inserting a finger into an upper of an interlock holder and into the inner of the finger lock holder;
a third process of changing into a ready state by pressing the interlock holder;
a fourth process of operating a laser module using a button module to generate laser beams, after the change into the ready state;
a fifth process of converging the generated laser beams into a focusing lens, passing a protect window, a strip hole, and an interlock holder, and then irradiating the laser beams into the inserted finger;
a sixth process of causing blood oozed from the finger due to the irradiating laser beams to be absorbed into the strip;
a seventh process of transmitting data regarding the absorbed blood to a controlling unit; and
an eighth process of outputting analyzed data regarding the absorbed blood from the controlling unit to a displaying unit.
